(19) United States
(12) Patent Application Publication
(10) Pub. No.: US 2023/0301836 A1
(43) Pub. Date: Sep. 28, 2023

(54) DRESSINGS HAVING SELECTABLE ADHESIVE FOR USE WITH INSTILLATION THERAPY AND NEGATIVE-PRESSURE THERAPY

(71) Applicant: KCI Manufacturing Unlimited Company, Westmeath (IE)

(72) Inventor: Christopher Brian LOCKE, Bournemouth (GB)

(21) Appl. No.: 18/020,307

(22) PCT Filed: Jul. 26, 2021
(86) PCT No.: PCT/IB2021/056740

§ 371 Date: Feb. 8, 2023

Related U.S. Application Data
(60) Provisional application No. 63/064,216, filed on Aug. 11, 2020.

(51) Int. Cl. A61F 13/02; A61F 13/00

(52) U.S. Cl. CPC A61F 13/00068; A61F 13/0203; A61M 1/90

(57) ABSTRACT

Dressings for treating a tissue site with instillation therapy are disclosed, which may include a dressing having a first layer comprising a polymer film having a plurality of fluid restrictions through the polymer film and a second layer comprising a polymer having a plurality of apertures. The second layer is adjacent to the first layer. The dressing may include an adhesive layer on at least a portion of the first layer. Further, the dressing may include a third layer on the adhesive layer, the third layer being at least partially removable from the adhesive layer so as to expose a portion of the adhesive.

### CROSS-REFERENCE TO RELATED APPLICATIONS

This application claims the benefit of priority to U.S. Provisional Application No. 63/064,216, filed on Aug. 11, 2020, which is incorporated herein by reference in its entirety.

### TECHNICAL FIELD

The invention set forth in the appended claims relates generally to tissue treatment systems and more particularly, but without limitation, to dressing materials that include selectable adhesive portions for use with negative-pressure therapy and instillation therapy

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation are widely known, improvements to therapy systems, components, and processes may benefit healthcare providers and patients.

### BRIEF SUMMARY

New and useful systems, apparatuses, and methods for treating tissue in a negative-pressure therapy or instillation therapy environment are set forth in the appended claims. Illustrative embodiments are also provided to enable a person skilled in the art to make and use the claimed subject matter.

For example, in some embodiments, a dressing for treating a tissue site with instillation therapy may comprise a first layer comprising a polymer film having a plurality of passages through the polymer film The second layer may comprise a polymer having a plurality of apertures. The second layer may be adjacent to the first layer. The dressing may also comprise an adhesive layer on at least a portion of the first layer, and a third layer on the adhesive layer. The third layer may be a liner that is at least partially removable from the adhesive layer. The third layer may be a non-adhesive third layer, and may comprise polyurethane. The third layer may include a plurality of fenestrations. Further, in some embodiments, the third layer may include a plurality of regions. The plurality of regions may be separable. The plurality of regions may be tessellate or concentric rings. Further, the plurality of regions may be separable along perforations between adjacent ones of the plurality of regions.

In further embodiments, a system for treating a tissue site may comprise a dressing and a source of instillation solution. The dressing may comprise a first layer comprising a polymer film having a plurality of fluid restrictions through the polymer film. The second layer may comprise a polymer having a plurality of apertures. The second layer may be adjacent to the first layer. The dressing may also comprise an adhesive layer on at least a portion of the first layer, and a third layer on the adhesive layer. The third layer may be at least partially removable from the adhesive layer. The third layer may be a non-adhesive third layer, which may comprise polyurethane and may include a plurality of fenestrations. Further, in some embodiments, the third layer may include a plurality of regions. The plurality of regions may be separable. The plurality of regions may be tessellate or concentric rings. Further, the plurality of regions may be separable along perforations between adjacent ones of the plurality of regions.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification.

FIG. 2 is an assembly view of an example of a dressing, illustrating additional details that may be associated with some example embodiments of the therapy system of FIG. 1.

FIG. 3 is a schematic view of the example layer of the dressing of FIG. 2.

FIG. 4 is an assembly view of another example of a dressing, illustrating additional details that may be associated with some example embodiments of the therapy system of FIG. 1.

FIG. 5 is a schematic view of an example configuration of apertures in a layer that may be associated with some embodiments of the dressing of FIG. 4.

FIG. 6 is a schematic view of the example layer of FIG. 5 overlaid on the example layer of FIG. 3.

FIG. 7 is an assembly view of another example of a dressing, illustrating additional details that may be associated with some example embodiments of the therapy system of FIG. 1.

FIG. 8 is a schematic view of an example configuration of apertures in a layer that may be associated with some embodiments of the dressing of FIG. 7.

FIG. 9 is a schematic view of the example layer of FIG. 8 overlaid on the example layer of FIG. 3.

FIG. 10 is an assembly view of a dressing that may be associated with an example embodiment of the therapy system of FIG. 1.

FIG. 11 is a top view of a manifold of the dressing of FIG. 10.

FIG. 12 is a cross-sectional view of the manifold of FIG. 11.

FIG. 13 is an assembly view of an example of a dressing that can be associated with some embodiments of the therapy system of FIG. 1.

FIG. 14 is a schematic view of an example layer that can be associated with some embodiments of the dressing of FIG. 13.

FIG. 15 is a side view of an example of the dressing of FIG. 14.

FIG. 16 is an assembly view of another example of a dressing that can be associated with some embodiments of the therapy system of FIG. 1.

FIG. 17 is a schematic view of an example layer that can be associated with some embodiments of the dressing of FIG. 16.

FIG. 18 is a schematic view of the example layer of FIG. 17 overlaid on the example layer of FIG. 14.

FIG. 19 is an assembly view of another example of a dressing that may be associated with some embodiments of the therapy system of FIG. 1.

FIG. 20 is a side, cross-sectional view of an example of a tissue interface that may be associated with some embodiments of the therapy system of FIG. 1.

FIG. 21 is an exploded side, cross-sectional view of another example of a tissue interface that may be associated with some example embodiments of the therapy system of FIG. 1.

FIG. 22 is an exploded side, cross-sectional view of another example of a tissue interface that may be associated with some example embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

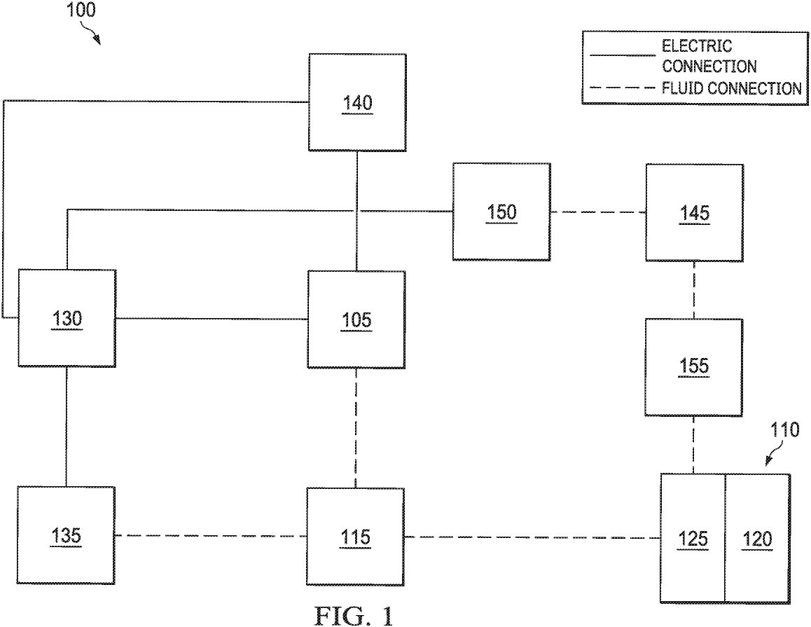

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness bums, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source, such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between -5 mm Hg (-667 Pa) and -500 mm Hg (-66.7 kPa). Common therapeutic ranges are between -50 mm Hg (-6.7 kPa) and -300 mm Hg (-39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface 120 may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface 120 may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3 M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic, silicone, or polyurethane adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

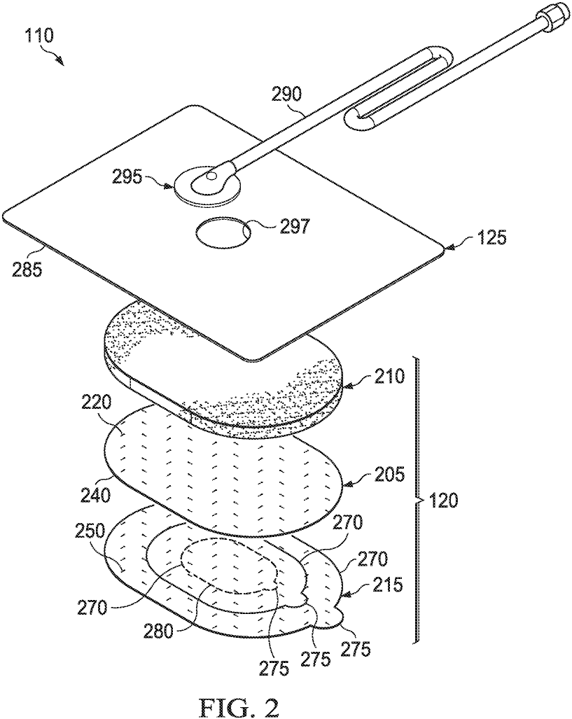

FIG. 2 is an assembly view of an example of the dressing 110 of FIG. 1, illustrating additional details that may be associated with some embodiments in which the tissue interface 120 comprises more than one layer. In the example of FIG. 2, the tissue interface 120 comprises a first layer 205, a second layer 210, and a third layer 215. In some embodiments, the first layer 205 may be disposed adjacent to the second layer 210, and the third layer 215 may also be disposed adjacent to the first layer 205. For example, the first layer 205 and the second layer 210 may be stacked so that the first layer 205 is in contact with the second layer 210. The first layer 205 may also be bonded to the second layer 210 in some embodiments. In some embodiments, the second layer 210 may be coextensive with a face of the first layer 205.

In some embodiments, at least some portion of the third layer 215 may be bonded to the first layer 205 by an adhesive 240. The adhesive 240 may be, for example, a medically-acceptable, pressure-sensitive adhesive that extends about a periphery, a portion, or the entire first layer 205. In some embodiments, for example, the adhesive 240 may be an acrylic adhesive having a coating weight between 25-65 grams per square meter (g.s.m.). In some embodiments, the adhesive 240 may be a polyurethane adhesive or a silicone adhesive. In some embodiments, the adhesive 240 may comprise or consist essentially of a sealing layer formed from a soft, pliable material, such as a tacky gel, suitable for providing a fluid seal with a tissue site, and may have a substantially flat surface. For example, the adhesive 240 may comprise, without limitation, a silicone gel, a soft silicone, hydrocolloid, hydrogel, polyurethane gel, polyolefin gel, hydrogenated styrenic copolymer gel, or a foamed gel. Thicker adhesives, or combinations of adhesives, may be applied in some embodiments to improve the seal and reduce leaks. In some embodiments, such a layer of the adhesive 240 may be continuous or discontinuous. Discontinuities in the adhesive 240 may be provided by apertures or holes (not shown) in the adhesive 240. The apertures or holes in the adhesive 240 may be formed after application of the adhesive 240 or by coating the adhesive 240 in patterns on a carrier layer, such as, for example, the first layer 205.

The first layer 205 may comprise or consist essentially of a means for controlling or managing fluid flow. In some embodiments, the first layer 205 may be a fluid control layer comprising or consisting essentially of a liquid-impermeable, elastomeric material. For example, the first layer 205 may comprise or consist essentially of a polymer film, such as a polyurethane film. In some embodiments, the first layer 205 may comprise or consist essentially of the same material as the cover 125. The first layer 205 may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the first layer 205 may have a substantially flat surface, with height variations limited to 0.2 millimeters over a centimeter.

In some embodiments, the first layer 205 may be hydrophobic. The hydrophobicity of the first layer 205 may vary, but may have a contact angle with water of at least ninety degrees in some embodiments. In some embodiments the first layer 205 may have a contact angle with water of no more than 150 degrees. For example, in some embodiments, the contact angle of the first layer 205 may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, VA, and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5-9 measured values, discarding both the highest and lowest measured values. The hydrophobicity of the first layer 205 may be further enhanced with a hydrophobic coating of other materials, such as silicones and fluorocarbons, either as coated from a liquid, or plasma coated.

The first layer 205 may also be suitable for welding to other layers, including the second layer 210. For example, the first layer 205 may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

The area density of the first layer 205 may vary according to a prescribed therapy or application. In some embodiments, an area density of less than 40 grams per square meter may be suitable, and an area density of about 20-30 grams per square meter may be particularly advantageous for some applications.

In some embodiments, for example, the first layer 205 may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

In some embodiments, the first layer 205 may include a polymer film of polylactic acid, carboxymethyl cellulose, or polycaprolactone. In other embodiments, the first layer 205 may include a film of xanthan gum mixed with at least one of collagen, oxidized regenerated cellulose, and alginate. In some embodiments, the first layer 205 includes a film of xanthan gum and citric acid mixed with at least one of collagen, oxidized regenerated cellulose, and alginate. The first layer 205 may include a film co-polymerized with dialkylcarbamoylchloride in some embodiments.

In some embodiments, the first layer 205 may be a film coated with petrolatum gel. The petrolatum gel may have a viscosity of at least 10000 millipascal seconds. In some embodiments, the petrolatum gel has anti-microbial compounds.

In some embodiments, instead of silicone and polyethylene films, the first layer 205 may include long residency bio-resorbably polymer film formed from polylactic acid, carboxymethylcellulose, polycaprolactone, or other polymers that are able to be cross-linked, such that function is retained for greater than about 7 days and resorption occurs in greater than 12 days. In other embodiments, the first layer may include highly cross-linked bioipolymers such as collagen or alginate, which are mixed with xanthan gum in a ratio of 20% gum to biologic, and which is plasma treated to achieve a hydrophobic in a desired ranged. The film may also include citric acid to assist with biofilm reduction and limit concerns with bacterial build-up. In some embodiments, the film is formed of polyethylene, polyurethane, EMA, or biopolymers incorporating a texture, such as “Sharklet” that assists with the reduction of biofilm formation on the dressing. In other embodiments, the film is co-polymerized with dialkylcarbamoylchloride, which is highly hydrophobic, and may aid in preventing biofilm and bacterial attachment.

The first layer 205 may have one or more passages, which can be distributed uniformly or randomly across the first layer 205. The passages may be bi-directional and pressure-responsive. For example, each of the passages generally may comprise or consist essentially of an elastic passage that is normally unstrained to substantially reduce liquid flow, and can expand or open in response to a pressure gradient. As illustrated in the example of FIG. 2, the passages may comprise or consist essentially of perforations 220 in the first layer 205. Perforations may be formed by removing material from the first layer 205. For example, perforations may be formed by cutting through the first layer 205. In the absence of a pressure gradient across the perforations, the perforations may be sufficiently small to form a seal or fluid restriction, which can substantially reduce or prevent liquid flow. Additionally, or alternatively, one or more of the passages may be or may function as an elastomeric valve that is normally closed when unstrained to substantially prevent liquid flow, and can open in response to a pressure gradient. In some examples, the passages may comprise or consist essentially of fenestrations in the first layer 205. Generally, fenestrations are a species of perforation, and may also be formed by removing material from the first layer 205. The amount of material removed and the resulting dimensions of the fenestrations may be up to an order of magnitude less than perforations.

In some embodiments, the perforations may be formed as slots, slits, or a combination of slots and slits in the first layer 205. In some examples, the perforations may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect elastomeric valves that can substantially reduce liquid flow in a normally closed or resting state. For example, such slots may form a flow restriction without being completely closed or sealed. The slots can expand or open wider in response to a pressure gradient to allow increased liquid flow.

The second layer 210 generally comprises or consists essentially of a manifold or a manifold layer, which provides a means for collecting or distributing fluid across the tissue interface 120 under pressure. For example, the second layer 210 may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as from a source of instillation solution, across the tissue interface 120.

In some illustrative embodiments, the pathways of the second layer 210 may be interconnected to improve distribution or collection of fluids. In some illustrative embodiments, the second layer 210 may comprise or consist essentially of a porous material having interconnected fluid pathways. Examples of suitable porous material that comprise or can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, the second layer 210 may additionally or alternatively comprise projections that form interconnected fluid pathways. For example, the second layer 210 may be molded to provide surface projections that define interconnected fluid pathways.

In some embodiments, the second layer 210 may comprise or consist essentially of a reticulated foam having pore sizes and free volume that may vary according to needs of a prescribed therapy. For example, a reticulated foam having a free volume of at least 90% may be suitable for many therapy applications, and a foam having an average pore size in a range of 400-600 microns may be particularly suitable for some types of therapy. The tensile strength of the second layer 210 may also vary according to needs of a prescribed therapy. For example, the tensile strength of a foam may be increased for instillation of topical treatment solutions. The 25% compression load deflection of the first layer 205 may be at least 0.35 pounds per square inch, and the 65% compression load deflection may be at least 0.43 pounds per square inch. In some embodiments, the tensile strength of the first layer 205 may be at least 10 pounds per square inch. The second layer 210 may have a tear strength of at least 2.5 pounds per inch. In some embodiments, the second layer 210 may be a foam comprised of polyols such as polyester or polyether, isocyanate such as toluene diisocyanate, and polymerization modifiers such as amines and tin compounds. In some examples, the first layer 205 may be a reticulated polyurethane foam such as used in GRANUFOAM™ dressing or V.A.C. VERAFLO™ dressing, both available from KCI of San Antonio, Texas.

Other suitable materials for the second layer 210 may include non-woven fabrics (Libeltex, Freudenberg), three-dimensional (3D) polymeric structures (molded polymers, embossed and formed films, and fusion bonded films [Supracore]), and mesh, for example.

In some examples, the second layer 210 may include a 3D textile, such as various textiles commercially available from Baltex, Muller, and Heathcoates. A 3D textile of polyester fibers may be particularly advantageous for some embodiments. For example, the second layer 210 may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 millimeters may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 millimeters in some embodiments. Such a spacer fabric may have a compression strength of about 20-25 kilopascals (at 40% compression). Additionally or alternatively, the second layer 210 may comprise or consist of a material having substantial linear stretch properties, such as a polyester spacer fabric having 2-way stretch and a weight of about 380 grams per square meter. A suitable spacer fabric may have a thickness of about 3-4 millimeters, and may have a warp and weft tensile strength of about 30-40 kilograms in some embodiments. The fabric may have a close-woven layer of polyester on one or more opposing faces in some examples. In some embodiments, a woven layer may be advantageously disposed on a second layer 210 to face a tissue site.

The second layer 210 generally has a first planar surface and a second planar surface opposite the first planar surface. The thickness of the second layer 210 between the first planar surface and the second planar surface may also vary according to needs of a prescribed therapy. For example, the thickness of the second layer 210 may be decreased to relieve stress on other layers and to reduce tension on peripheral tissue. The thickness of the second layer 210 can also affect the conformability of the second layer 210. In some embodiments, a suitable foam may have a thickness in a range of about 5 millimeters to 10 millimeters. Fabrics, including suitable 3D textiles and spacer fabrics, may have a thickness in a range of about 2 millimeters to about 8 millimeters.

The third layer 215 may be a release liner and may be at least partially removable so as to expose at least a portion of the adhesive 240 on the first layer 205. The third layer 215 may also provide stiffness to assist with, for example, deployment of the dressing 110. The third layer 215 may be, for example, a casting paper, a film, or polyethylene. Further, in some embodiments, the third layer 215 may be a polyester material such as polyethylene terephthalate (PET), or similar polar semi-crystalline polymer. The use of a polar semi-crystalline polymer for the third layer 215 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. Further, a release agent may be disposed on a side of the third layer 215 that is configured to contact the first layer 205. For example, the release agent may be a silicone coating and may have a release factor suitable to facilitate removal of the third layer 215 from the adhesive 240 by hand and without damaging or deforming the dressing 110. In some embodiments, the release agent may be a fluorocarbon or a fluorosilicone, for example. In other embodiments, the third layer 215 may be uncoated or otherwise used without a release agent.

In some embodiments, the third layer 215 is formed of polyurethane and includes one or more passages 250, which can be distributed uniformly or randomly across the third layer 215, and can restrict fluid transfer across or through the third layer 215. The passages 250 are aligned with the perforations in the first layer 205. In some embodiments, the passages 250 may be fluid restrictions. The passages 250 may be bi-directional and pressure-responsive. For example, each of the passages 250 generally may comprise or consist essentially of an elastic passage that is normally unstrained to substantially reduce liquid flow, and can expand or open in response to a pressure gradient. In some embodiments, the passages 250 may comprise or consist essentially of perforations in the third layer 215. Perforations may be formed by removing material from the third layer 215. For example, perforations may be formed by cutting through the third layer 215, which may also deform the edges of the perforations in some embodiments. The perforations may be about 3 mm long and about 0.8 mm wide in some embodiments. In the absence of a pressure gradient across the perforations, the passages may be sufficiently small to form a seal or fluid restriction, which can substantially reduce or prevent liquid flow. Additionally or alternatively, one or more of the passages 250 may be an elastomeric valve that is normally closed when unstrained to substantially prevent liquid flow, and can open in response to a pressure gradient. A fenestration in the third layer 215 may be a suitable valve for some applications. Fenestrations may also be formed by removing material from the third layer 215, but the amount of material removed and the resulting dimensions of the fenestrations may be up to an order of magnitude less than perforations, and may not deform the edges. Accordingly, the third layer 215 may be a perforated release liner.

For example, some embodiments of the passages 250 may comprise or consist essentially of one or more fenestrations, perforations, or combinations of fenestrations and perforations in the third layer 215. In some examples, the passages 250 may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In some embodiments, the third layer 215 may include a plurality of separable regions 270, such that one or more of the separable regions 270 may be removed. Thus, only a portion of the adhesive 240 on the first layer 205 may be exposed where, for example, only one of the plurality of separable regions 270 is removed. As illustrated in the example of FIG. 2, the plurality of separable regions 270 may be concentric rings or ovals in some embodiments. In other examples, the separable regions 270 may be configured as tessellate pattern. The plurality of separable regions 270 may be separated by perforations 280 to enable easy removal of one or more of the plurality of separable regions 270. Thus, one or more of the concentric rings or ovals may be removed, such that only a generally ring-shaped portion of the adhesive 240 is exposed. The third layer 215 and/or each of the plurality of separable regions 270 may also include a pull tab 275 to allow for easy removal of the third layer 215 or at least one of the plurality of separable regions 270 so as to expose at least a portion of the adhesive 240.

As illustrated in the example of FIG. 2, the dressing 110 may further include an attachment device, such as an adhesive 285. The adhesive 285 may be, for example, a medically-acceptable, pressure-sensitive adhesive that extends about a periphery, a portion, or an entire surface of the cover 125. In some embodiments, for example, the adhesive 285 may be an acrylic adhesive having a coating weight between 25-65 grams per square meter (g.s.m.). Thicker adhesives, or combinations of adhesives, may be applied in some embodiments to improve the seal and reduce leaks. In some embodiments, such a layer of the adhesive 285 may be continuous or discontinuous. Discontinuities in the adhesive 285 may be provided by apertures or holes (not shown) in the adhesive 285. The apertures or holes in the adhesive 285 may be formed after application of the adhesive 285 or by coating the adhesive 285 in patterns on a carrier layer, such as, for example, a side of the cover 125. Apertures or holes in the adhesive 285 may also be sized to enhance the MVTR of the dressing 110 in some example embodiments.

FIG. 2 also illustrates one example of a fluid conductor 290 and a dressing interface 295. As shown in the example of FIG. 2, the fluid conductor 290 may be a flexible tube, which can be fluidly coupled on one end to the dressing interface 295. The dressing interface 295 may be an elbow connector, as shown in the example of FIG. 2, which can be placed over an aperture 297 in the cover 125 to provide a fluid path between the fluid conductor 290 and the tissue interface 120.

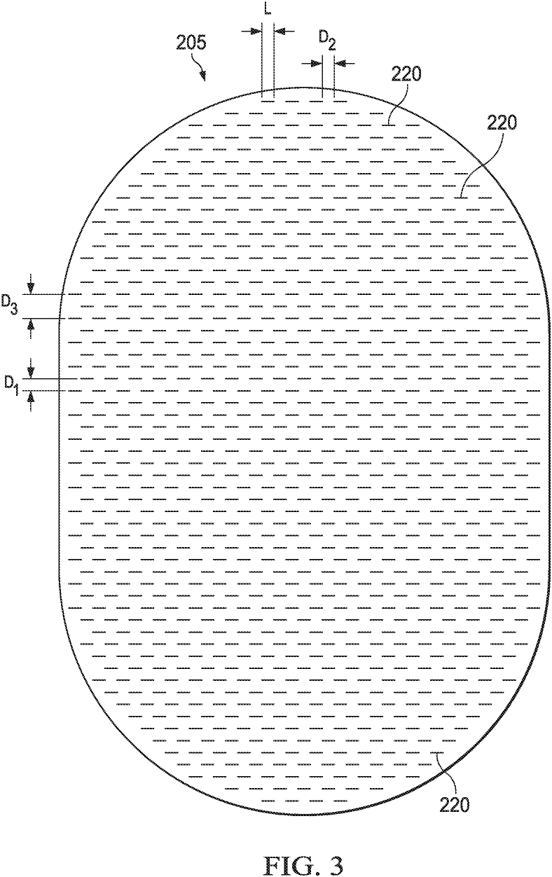

FIG. 3 is a schematic view of an example of the first layer 205, illustrating additional details that may be associated with some embodiments. As illustrated in the example of FIG. 3, the perforations 220 may each consist essentially of one or more linear slots, fenestrations, or perforations having a length L. A length L of about 3 millimeters may be suitable for some examples. FIG. 3 additionally illustrates an example of a uniform distribution pattern of the perforations 220. In FIG. 3, the perforations 220 are substantially coextensive with the first layer 205, and are distributed across the first layer 205 in a grid of parallel rows and columns, in which the perforations 220 are also mutually parallel to each other. The rows may be spaced a distance D1, and the perforations 220 within each of the rows may be spaced a distance D2. For example, a distance D1 of about 3 millimeters on center and a distance D2 of about 3 millimeters may be suitable for some embodiments. The perforations 220 in adjacent rows may be aligned or offset. For example, adjacent rows may be offset, as illustrated in FIG. 3, so that the perforations 220 are aligned in alternating rows separated by a distance D3. A distance D3 of about 6 millimeters may be suitable for some examples. The spacing of the perforations 220 may vary in some embodiments to increase the density of the perforations 220 according to therapeutic requirements.

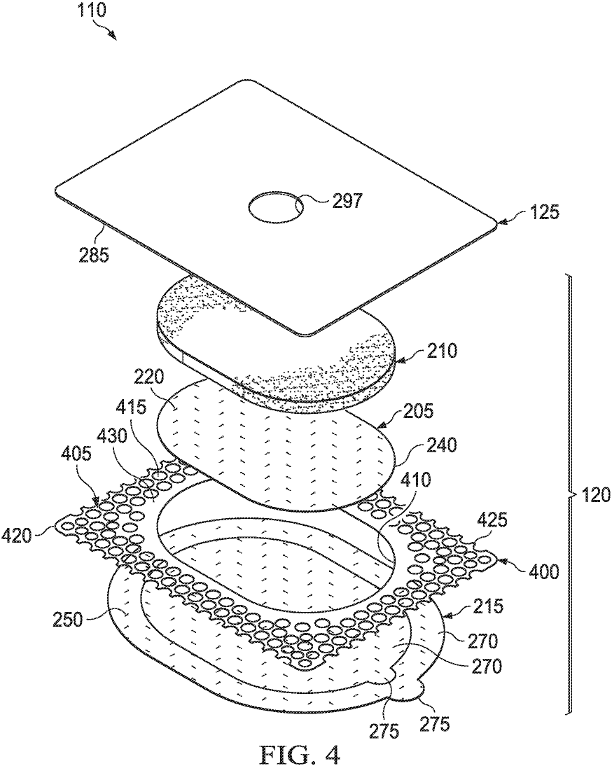

FIG. 4 is an assembly view of another example of the dressing 110 of FIG. 1, illustrating additional details that may be associated with some embodiments. In some embodiments, as shown in FIG. 4, the tissue interface 120 may also include a fourth layer 400. The fourth layer 400 may comprise or consist essentially of a sealing layer formed from a soft, pliable material suitable for providing a fluid seal with a tissue site, such as a suitable gel material, and may have a substantially flat surface. For example, the fourth layer 400 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In some embodiments, the fourth layer 400 may have a thickness between about 200 microns (µm) and about 1000 microns (µm). In some embodiments, the fourth layer 400 may have a hardness between about 5 Shore OO and about 80 Shore OO. Further, the fourth layer 400 may be comprised of hydrophobic or hydrophilic materials.

In some embodiments, the fourth layer 400 may be a hydrophobic-coated material. For example, the fourth layer 400 may be formed by coating a spaced material, such as, for example, woven, nonwoven, molded, or extruded mesh with a hydrophobic material. The hydrophobic material for the coating may be a soft silicone, for example.

The fourth layer 400 may have a periphery 405 surrounding or around a treatment aperture 410, and apertures 415 in the periphery 405 disposed around the treatment aperture 410. The treatment aperture 410 may be complementary or correspond to a surface area of the first layer 205 in some examples. For example, the treatment aperture 410 may form a frame, window, or other opening around a surface of the first layer 205. The fourth layer 400 may also have corners 420 and edges 425. The comers 420 and the edges 425 may be part of the periphery 405. The fourth layer 400 may have an interior border 430 around the treatment aperture 410, which may be substantially free of the apertures 415, as illustrated in the example of FIG. 4. In some examples, as illustrated in FIG. 2, the treatment aperture 410 may be symmetrical and centrally disposed in the fourth layer 400, forming an open central window.

The apertures 415 may be formed by cutting, perforating, or by application of local RF or ultrasonic energy, for example, or by other suitable techniques for forming an opening or perforation in the fourth layer 400. The apertures 415 may have a uniform distribution pattern, or may be randomly distributed on the fourth layer 400. The apertures 415 in the fourth layer 400 may have many shapes, including circles, squares, stars, ovals, polygons, slits, complex curves, rectilinear shapes, triangles, for example, or may have some combination of such shapes.

Each of the apertures 415 may have uniform or similar geometric properties. For example, in some embodiments, each of the apertures 415 may be circular apertures, having substantially the same diameter. In some embodiments, each of the apertures 415 may have a diameter of about 1 millimeter to about 50 millimeters. In other embodiments, the diameter of each of the apertures 415 may be about 1 millimeter to about 20 millimeters.

In other embodiments, geometric properties of the apertures 415 may vary. For example, the diameter of the apertures 415 may vary depending on the position of the apertures 415 in the fourth layer 400. For example, in some embodiments, the apertures 415 disposed in the periphery 405 may have a diameter between about 5 millimeters and about 10 millimeters. A range of about 7 millimeters to about 9 millimeters may be suitable for some examples. In some embodiments, the apertures 415 disposed in the corners 420 may have a diameter between about 7 millimeters and about 8 millimeters.

At least one of the apertures 415 in the periphery 405 of the fourth layer 400 may be positioned at the edges 425 of the periphery 405, and may have an interior cut open or exposed at the edges 425 that is in fluid communication in a lateral direction with the edges 425. The lateral direction may refer to a direction toward the edges 425 and in the same plane as the fourth layer 400. As shown, the apertures 415 in the periphery 405 may be positioned proximate to or at the edges 425 and in fluid communication in a lateral direction with the edges 425. The apertures 415 positioned proximate to or at the edges 425 may be spaced substantially equidistant around the periphery 405 as shown in the example of FIG. 4. Alternatively, the spacing of the apertures 415 proximate to or at the edges 425 may be irregular.

As illustrated in the example of FIG. 4, in some embodiments, the dressing 110 may include the third layer 215 to protect the adhesive 240 prior to use. The third layer 215 includes the plurality of separable regions 270, such that one or more of the plurality of separable regions 270 may be removed to expose some or all of the adhesive 240. The plurality of separable regions 270 of the third layer 215 may have a shape similar to the first layer 205. Further, the plurality of separable regions 270 may include a plurality of rings. An outer ring of the plurality of rings may not include passages 250 therein.

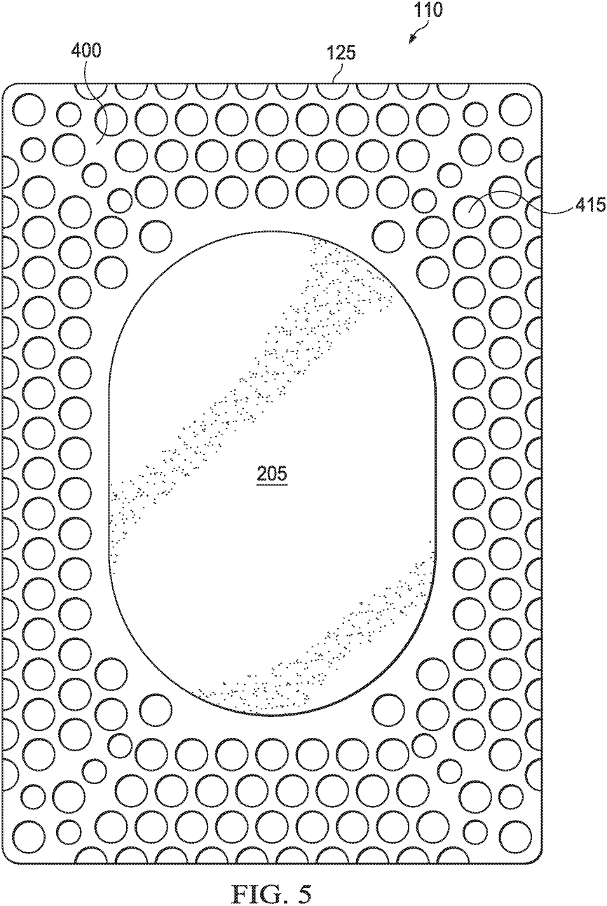

FIG. 5 is a top view of the dressing 110 in the example of FIG. 4, as assembled, illustrating additional details that may be associated with some embodiments. As illustrated in the example of FIG. 5, the cover 125 and the fourth layer 400 may have substantially the same perimeter shape and dimensions, so that the cover 125 and the fourth layer 400 are coextensive in some examples. The cover 125 may be substantially transparent, allowing visibility of the apertures 415 in some embodiments. The first layer 205 may be centrally disposed over the fourth layer 400, such as over the treatment aperture 230 (not visible in FIG. 5). The cover 125 may be disposed over the first layer 205 and coupled to the fourth layer 400 around the first layer 205 so that at least some of the adhesive 285 can be disposed adjacent to the apertures 415.

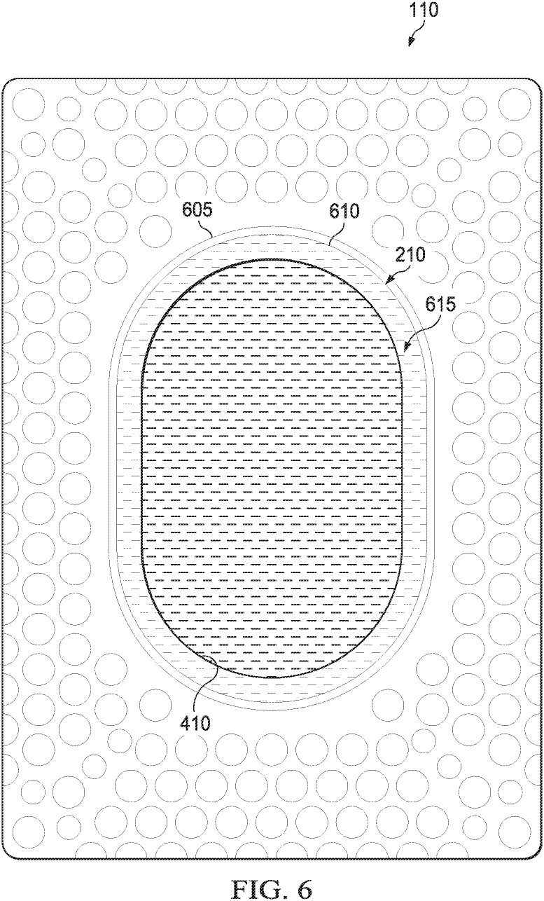

FIG. 6 is a bottom view of the dressing 110 in the example of FIG. 4, as assembled and with the third layer 215 removed, illustrating additional details that may be associated with some embodiments. As illustrated in the example of FIG. 6, a substantial number of the perforations 220 may be aligned or otherwise exposed through the treatment aperture 410, and at least some portion of the first layer 205 may be disposed adjacent to the perforations 220 opposite the treatment aperture 410. In some embodiments, the first layer 205 and the second layer 210 may be substantially aligned with the treatment aperture 410, or may extend across the treatment aperture 230.

Additionally, the first layer 205 may have a first edge 605, and the second layer 210 may have a second edge 610. In some examples, the first edge 605 and the second edge 610 may have substantially the same shape so that adjacent faces of the first layer 205 and the second layer 210 are geometrically similar. The first edge 605 and the second edge 610 may also be congruent in some examples, so that adjacent faces of the first layer 205 and the second layer 210 are substantially coextensive and have substantially the same surface area. In the example of FIG. 6, the first edge 605 defines a larger face of the first layer 205 than the face of the second layer 210 defined by the second edge 610, and the larger face of the first layer 205 extends past the smaller face of the second edge 610.

The faces defined by the first edge 605, the second edge 610, or both may also be geometrically similar to the treatment aperture 410 in some embodiments, as illustrated in the example of FIG. 6, and may be larger than the treatment aperture 410. The fourth layer 400 may have an overlay margin 615 around the treatment aperture 410, which may have an additional adhesive disposed therein. As illustrated in the example of FIG. 6, the treatment aperture 410 may be an ellipse or a stadium in some embodiments. The treatment aperture 410 may have an area that is equal to about 20% to about 80% of the area of the fourth layer 400 in some examples The treatment aperture 410 may also have an area that is equal to about 20% to about 80% of the area of a face of defined by the first edge 605 of the first layer 205. A width of about 90 millimeters to about 110 millimeters and a length of about 150 millimeters to about 160 millimeters may be suitable for some embodiments of the treatment aperture 230. For example, the width of the treatment aperture 230 may be about 100 millimeters, and the length may be about 155 millimeters. In some embodiments, a suitable width for the overlay margin 615 may be about 2 millimeters to about 3 millimeters. For example, the overlay margin 615 may be coextensive with an area defined between the treatment aperture 410 and the first edge 605, and the adhesive may secure the first layer 205, the second layer 210, or both to the third layer 215 and/or the fourth layer 400.

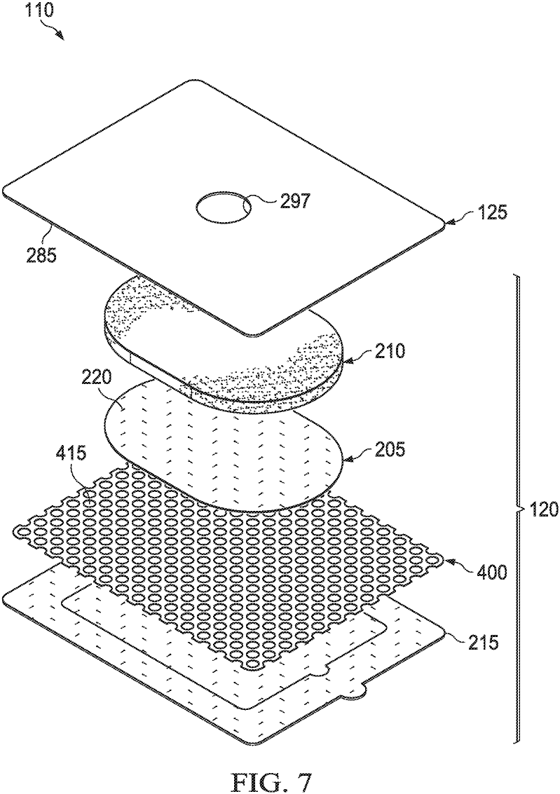

FIG. 7 is an assembly view of another example of the dressing 110 of FIG. 1, illustrating additional details that may be associated with some embodiments. As illustrated in FIG. 7, some examples of the fourth layer 400 may not have the treatment aperture 410, and the apertures 415 may be distributed in a uniform pattern across the fourth layer 400. In some embodiments, one or more of the cover 125, the third layer 215, and the fourth layer 400 may also be congruent in some examples, so that adjacent faces of one or more of the cover 125, the third layer 215, and the fourth layer 400 are substantially coextensive.

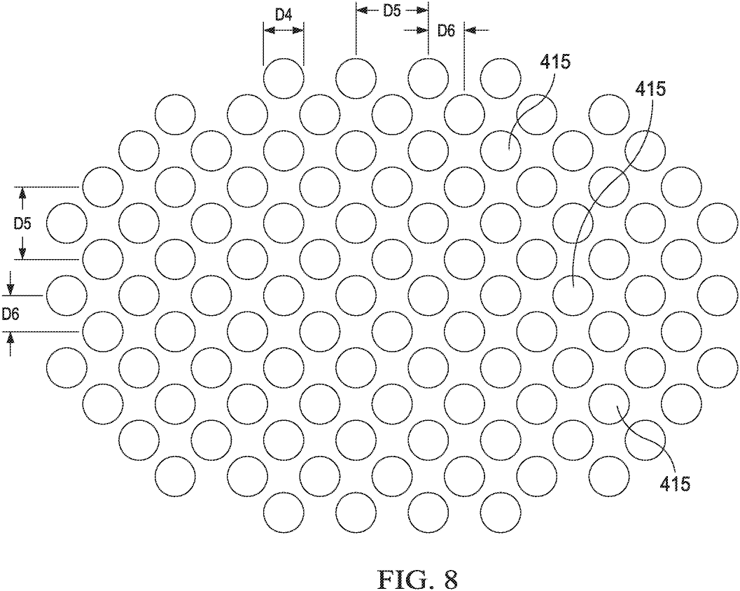

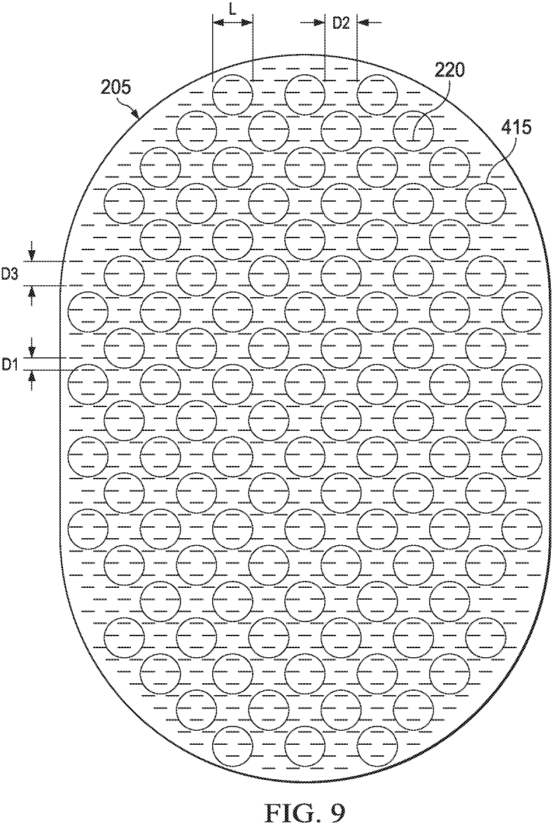

FIG. 8 is a schematic view of an example configuration of the apertures 415, illustrating additional details that may be associated with some embodiments of the fourth layer 400. In the example of FIG. 8, the apertures 415 are generally circular and have a diameter D4, which may be about 6 millimeters to about 8 millimeters in some embodiments. A diameter D4 of about 7 millimeters may be particularly suitable for some embodiments. FIG. 9 also illustrates an example of a uniform distribution pattern of the apertures 415. In FIG. 9, the apertures 415 are distributed across the fourth layer 400 in a grid of parallel rows and columns. Within each row and column, the apertures 415 may be equidistant from each other, as illustrated in the example of FIG. 8. FIG. 8 illustrates one example configuration that may be particularly suitable for many applications, in which the apertures 415 are spaced a distance D5 apart along each row and column, with an offset of D6. In some examples, the distance D5 may be about 9 millimeters to about 10 millimeters, and the offset D6 may be about 8 millimeters to about 9 millimeters.

FIG. 9 is a schematic view of the apertures 415 in the example of FIG. 8 overlaid on the first layer 205 of FIG. 3, illustrating additional details that may be associated with some example embodiments of the tissue interface 120. For example, as illustrated in FIG. 9, more than one of the perforations 220 may be aligned, overlapping, in registration with, or otherwise fluidly coupled to the apertures 415 in some embodiments. In some embodiments, one or more of the perforations 220 may be only partially registered with the apertures 415. The apertures 415 in the example of FIG. 9 are generally sized and configured so that at least four of the perforations 220 is registered with each one of the apertures 415. In other examples, one or more of the perforations 220 may be registered with more than one of the apertures 415. For example, any one or more of the perforations 220 may be a perforation or a fenestration that extends across two or more of the apertures 415. Additionally or alternatively, one or more of the perforations 220 may not be registered with any of the apertures 415.

As illustrated in the example of FIG. 9, the apertures 415 may be sized to expose a portion of the first layer 205, the perforations 220, or both through the fourth layer 400. The apertures 415 in the example of FIG. 9 are generally sized to expose more than one of the perforations 220. Some or all of the apertures 415 may be sized to expose two or three of the perforations 220. In some examples, the length of each of the perforations 220 may be substantially smaller than the diameter of each of the apertures 415. More generally, the average dimensions of the perforations 220 are substantially smaller than the average dimensions of the apertures 415. In some examples, the apertures 415 may be elliptical, and the length of each of the perforations 220 may be substantially smaller than the major axis or the minor axis. In some embodiments, though, the dimensions of the perforations 220 may exceed the dimensions of the apertures 415, and the size of the apertures 415 may limit the effective size of the perforations 220 exposed to the lower surface of the dressing 110.

Individual components of the dressing 110 in the examples of FIGS. 4-9 may be bonded or otherwise secured to one another with a solvent or non-solvent adhesive, or with thermal welding, for example, without adversely affecting fluid management.

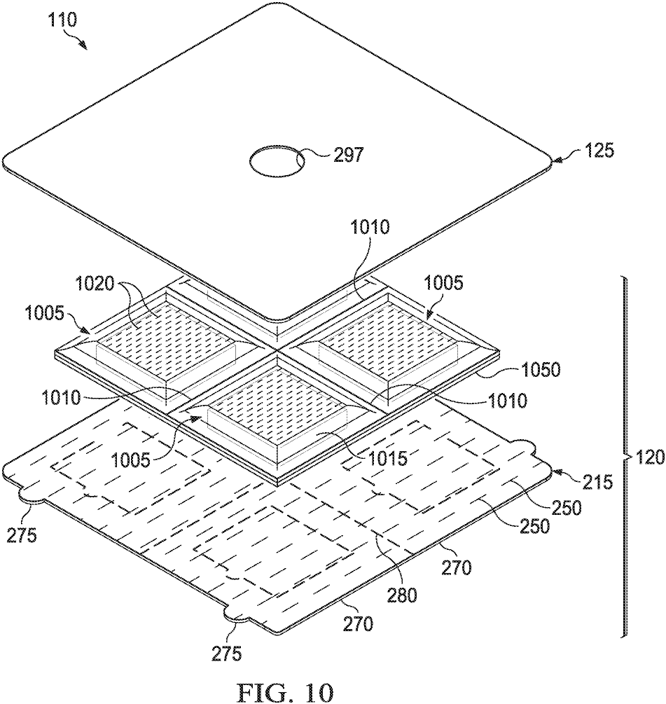

FIG. 10 is an assembly view of another example embodiment of the dressing 110 of FIG. 1, illustrating additional details that may be associated with some embodiments in which the tissue interface 120 comprises separable sections. In the example of FIG. 10, the tissue interface 120 comprises one or more separable sections 1005, which may be bounded by seams 1010. Each of the separable sections 1005 may include a manifold section 1015. In some examples, seams 1010 may be formed between or may define the manifold sections 1015.

The manifold sections 1015 may comprise or consist of foam in some embodiments. For example, the foam may be an open-cell foam, such as reticulated foam. The foam may also be relatively thin and hydrophobic to reduce the fluid hold capacity of the dressing, which can encourage exudate and other fluid to pass quickly to external storage. The foam layer may also be thin to reduce the dressing profile and increase flexibility, which can enable it to conform to wound beds and other tissue sites under negative pressure. In some embodiments, the manifold sections 1015 may be formed of 3-dimensional textiles, non-woven wicking material, vacuum-formed texture surfaces, and composites thereof. A hydrophobic manifold having a thickness of less than 7 millimeters and a free volume of at least 90% may be suitable for many therapeutic applications. In some embodiments, the manifold sections 1015 may be formed of colored material. Each of the manifold sections 1015 may be a same color or a different color.

As illustrated in the example of FIG. 10, the tissue interface 120 may have one or more passages 1020, such as fluid restrictions, which can be distributed uniformly or randomly across the tissue interface 120. The passages 1020 may be bi-directional and pressure-responsive. For example, each of the passages 1020 generally may comprise or consist essentially of an elastic passage that is normally unstrained to substantially reduce liquid flow, and can expand or open in response to a pressure gradient. The passages 1020 may be coextensive with the manifold sections 1015.

For example, some embodiments of the passages 1020 may comprise or consist essentially of one or more slits, slots or combinations of slits and slots. In some examples, the passages 1020 may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. In some embodiments, the fluid restrictions 1120 may be formed by ultrasonics or other heat means. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In some embodiments, an adhesive 1050 is applied to at least one surface of the tissue interface 120. The adhesive 1050 may be, for example, a medically-acceptable, pressure-sensitive adhesive that extends about a periphery, a portion, or the entire tissue interface 120 as described herein

As illustrated in the example of FIG. 10, in some embodiments, the dressing 110 may include the third layer 215 to protect the adhesive 1050 prior to use and/or during use. The third layer 215 may include a plurality of separable regions 270 associated with each of the separable sections 1005, such that when the separable sections 1005 are separated, all or a portion of the third layer 215 of the separated separable section 1005 can be removed so as to expose at least a portion of the adhesive 1050.

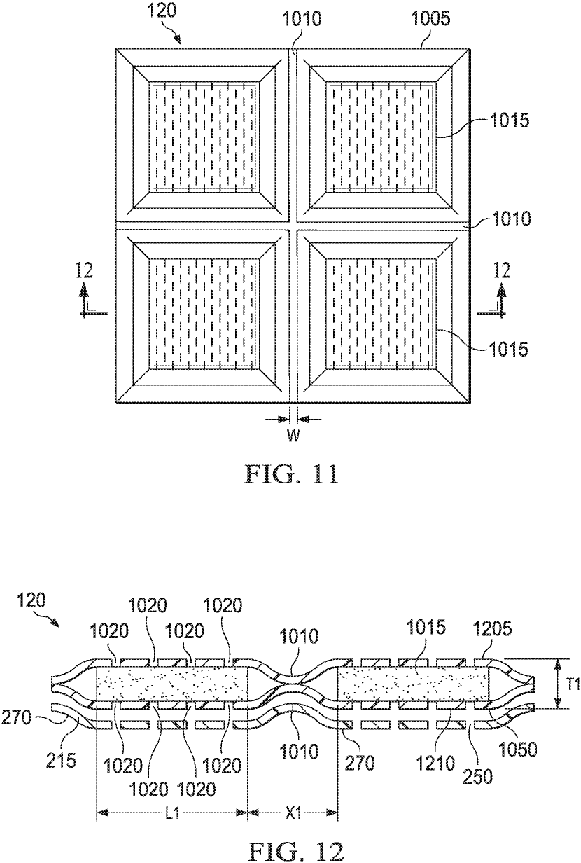

FIG. 11 is a top view of the tissue interface 120 of FIG. 10, illustrating additional details that may be associated with some examples. The manifold sections 1015 in each of the separable sections 1005 may have a same shape or a different shape. As shown in the example of FIG. 11, the separable sections 1005 and the manifold sections 1015 may have similar shapes. In some embodiments, each of the separable sections 1005 and the manifold sections 1015 may have a tessellate shape, such as the generally square shape in the example of FIG. 11, with sides having a length ranging from about 10 mm to about 30 mm (e.g., about 15 mm to about 25 mm or about 18 mm to about 22 mm). For example, the manifold sections 1015 may be squares having dimensions of about 20 mm by about 20 mm.

Each of the seams 1010 may have a width W ranging from about 2 mm to about 5 mm, and may be wide enough to allow for the separable sections 1005 to be separated along the seams 1010 without exposing any portion of the manifold sections 1015.

FIG. 12 is a section view of the tissue interface 120 of FIG. 11 taken along line 12-12, illustrating additional details that may be associated with some embodiments. In the example of FIG. 12, the tissue interface 120 comprises a first film layer 1205, a second film layer 1210, and the manifold sections 1015 disposed between the first film layer 1205 and the second film layer 1210. In some embodiments, the first film layer 1205 and the second film layer 1210 may be disposed adjacent to the manifold sections 1015 as shown in the example of FIG. 12. Also as shown in the example of FIG. 12, the seams 1010 may be formed by one or more bonds between the first film layer 1205 and the second film layer 1210. The bonds may be continuous or discrete.

The first film layer 1205 and the second film layer 1210 may comprise or consist essentially of a means for controlling or managing fluid flow. In some embodiments, the first film layer 1205 and the second film layer 1210 may comprise or consist essentially of an elastomeric material that is impermeable to liquid. For example, the first film layer 1205 and the second film layer 1210 may comprise or consist essentially of a polymer film. The first film layer 1205 and the second film layer 1210 may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the second layer may have a substantially flat surface, with height variations limited to 0.2 millimeters over a centimeter.

In some embodiments, the first film layer 1205 and the second film layer 1210 may comprise or consist essentially of a hydrophobic material. The hydrophobicity may vary, but may have a contact angle with water of at least ninety degrees in some embodiments. In some embodiments the hydrophobic material may have a contact angle with water of no more than 150 degrees. For example, in some embodiments, the contact angle may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, VA, and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of the first film layer 1205, the second film layer 1210, or both may be further enhanced with a hydrophobic coating of other materials, such as silicones and fluorocarbons, either as coated from a liquid, or plasma coated.

The first film layer 1205 and the second film layer 1210 may also be suitable for bonding to other layers, including each other. For example, the first film layer 1205, the second film layer 1210, or both may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene. The first film layer 1205 and the second film layer 1210 may include hot melt films.

The area density of the first film layer 1205 and the second film layer 1210 may vary according to a prescribed therapy or application. In some embodiments, an area density of less than 40 grams per square meter may be suitable, and an area density of about 20-30 grams per square meter may be particularly advantageous for some applications.

In some embodiments, for example, the first film layer 1205, the second film layer 1210, or both may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

In some embodiments, the passages 1020 may comprise or consist essentially of perforations in at least one of the first film layer 1205 and the second film layer 1210. Perforations may be formed by removing material from the first film layer 1205, the second film layer 1210, or both. For example, perforations may be formed by cutting through the material, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the passages 1020 may be an elastomeric valve that is normally closed when unstrained to substantially prevent liquid flow, and can open in response to a pressure gradient. A fenestration in the material may be a suitable valve for some applications. Fenestrations may also be formed by removing material, but the amount of material removed and the resulting dimensions of the fenestrations may be an order of magnitude less than perforations, and may not deform the edges. In some embodiments, the passages 1020 extend through both the first film layer 1205 and the second film layer 1210, and the passages 1020 are coextensive with at least one of the first film layer 1205 and the second film layer 1210.

Each of the manifold sections 1015 has a length L1, which can be in a range from about 10 mm to about 30 mm (e.g., about 15 mm to about 25 mm or about 18 mm to about 22 mm). For example, each of the manifold sections 1015 may have a length of about 20 mm. In some embodiments, the manifold sections 1015 may be spaced apart by a distance X1 of about 5 mm to about 15 mm. For example, a distance X1 of about 10 mm may be particularly advantageous for some embodiments.

In some embodiments, each of the manifold sections 1015 in the tissue interface 120 may be the same size. In other embodiments, one or more of the manifold sections 1015 in the tissue interface 120 may have a different size.

In some embodiments, the tissue interface 120 has a thickness T1 ranging from about 5 mm to about 20 mm (e.g., about 8 mm to about 18 mm, or about 10 mm to about 15 mm). For example, the tissue interface 120 may have a thickness T1 of about 8 mm. The thickness T1 of the tissue interface 120 may vary depending upon a thickness of the manifold sections 1015 used to form the tissue interface 120. For example, each of the manifold sections 1015 may have a thickness ranging from about 5 mm to about 15 mm (e.g., about 8 mm to about 12 mm).

In some embodiments, the first layer 1205 and the second layer 1210 may be formed of a transparent polymer to aid in cutting the separable sections 1005 apart along the seams 1010.

In some embodiments, the tissue interface 120 can be formed by spacing the manifold sections 1015 apart, placing the first layer 1205 of polymer film over the manifold sections 1015, placing the second layer 1210 under the manifold sections 1015, and bonding the first layer 1205 to the second layer 1210, forming the seams 1010 between the manifold sections 1015. Suitable means for bonding the first layer 1205 to the second layer 1210 may include, for example, an adhesive such as an acrylic, and welding, such as heat, radio frequency (RF), or ultrasonic welding. In some embodiments, sacrificial materials may be disposed between the first layer 1205 and the second layer 1210 to facilitate welding. Suitable sacrificial materials may include, for example, hot melt films supplied by Bayer (such as H2, HU2, and H5 films), Comelius (Collano film), or Prochimir (such as TC203 or TC206 film).

In some embodiments, the manifold sections may be formed from an integral manifold material, such as foam. In some embodiments, for example, bonds between the first layer 1205 and the second layer 1210 may extend through a layer of manifold material to define the manifold sections 1015. For example, some embodiments of a manifold layer may have a thickness ranging from about 5 mm to about 8 mm, and at least one of the first layer 1205 and the second layer 1210 may melt through the manifold layer during welding to form the seams 1010.

Additionally or alternatively, a unitary manifold material can be perforated and cut to define the manifold sections 1015 in a variety of suitable shapes and patterns. In some embodiments, the seams 1010 may align with perforations between the manifold sections 1015. In some examples, sacrificial joints may be left between the manifold sections 1015 to maintain the manifold sections 1015 together as a single unit. Maintaining the manifold sections 1015 as a single unit can allow for easier assembly of the tissue interface 120. In some embodiments, either or both of the first layer 1205 and the second layer 1210 may also be bonded to the manifold sections 1015 for additional stability.

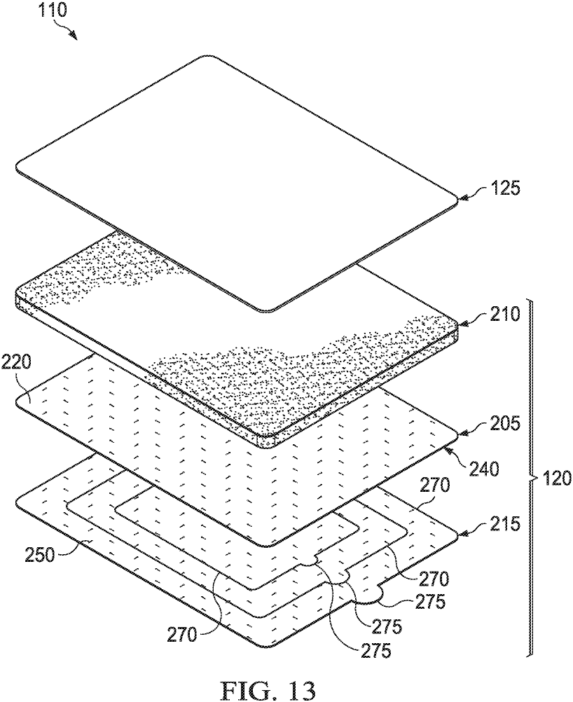

FIG. 13 is an assembly view of another example of the dressing 110 of FIG. 1, illustrating additional details that may be associated with some embodiments in which the tissue interface 120 comprises more than one layer. In the example of FIG. 13, the tissue interface 120 comprises the first layer 205, the second layer 210, and the third layer 215. In some embodiments, the first layer 205 may be disposed adjacent to the second layer 210. For example, the first layer 205 and the second layer 210 may be stacked so that the first layer 205 is in contact with the second layer 210. The first layer 205 may also be heat-bonded or adhered to the second layer 210 in some embodiments. In some embodiments, the first layer 205 optionally includes the adhesive 240, such as a low-tack adhesive or an acrylic adhesive. The adhesive 240 may be continuously coated on the first layer 205 or applied in a pattern.

The first layer 205 may comprise or consist essentially of a means for controlling or managing fluid flow. In some embodiments, the first layer 205 may be a fluid control layer comprising or consisting essentially of a liquid-impermeable, elastomeric material. For example, the first layer 205 may comprise or consist essentially of a polymer film, such as a polyurethane film as described herein. In some embodiments, the first layer 205 may comprise or consist essentially of the same material as the cover 125. The first layer 205 may also have a smooth or matte surface texture in some embodiments. A glossy or shiny finish finer or equal to a grade B3 according to the SPI (Society of the Plastics Industry) standards may be particularly advantageous for some applications. In some embodiments, variations in surface height may be limited to acceptable tolerances. For example, the surface of the first layer 205 may have a substantially flat surface, with height variations limited to 0.2 millimeters over a centimeter.

In some embodiments, as shown in FIG. 13, the tissue interface 120 including the third layer 215, can be substantially rectangular in shape. Further, each of the plurality of separable regions 270 of the third layer 215 can be rectangular in shape.

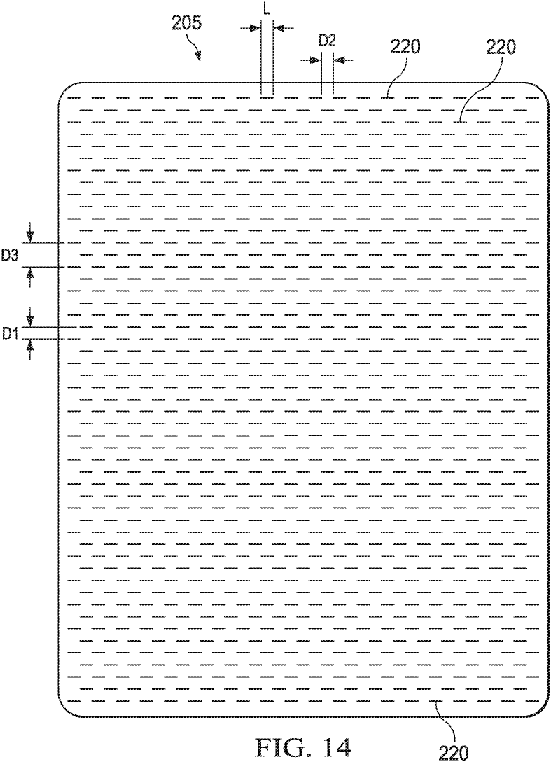

FIG. 14 is a schematic view of another example of the first layer 205, illustrating additional details that may be associated with some embodiments. As illustrated in the example of FIG. 14, the first layer 205 may have a rectangular shape.

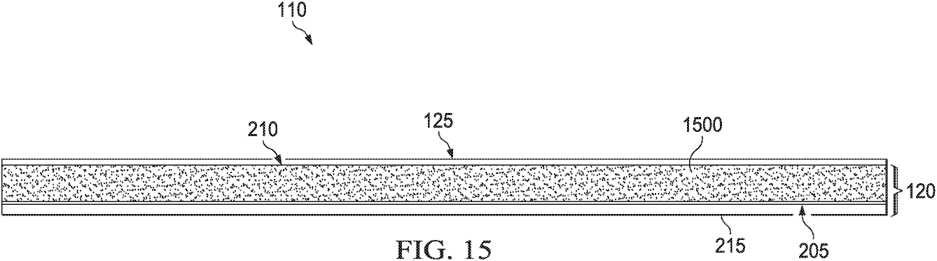

FIG. 15 is a side view of an example of the dressing 110 of FIG. 13 that may be associated with some embodiments of the therapy system of FIG. 1. As shown in FIG. 15, the tissue interface 120 has an exposed perimeter 1500. More particularly, in the example of FIG. 15, the cover 125, the first layer 205, the second layer 210, and the third layer 215 each have an exposed perimeter, and there is no seam, weld, or seal along the exposed perimeter 1600.

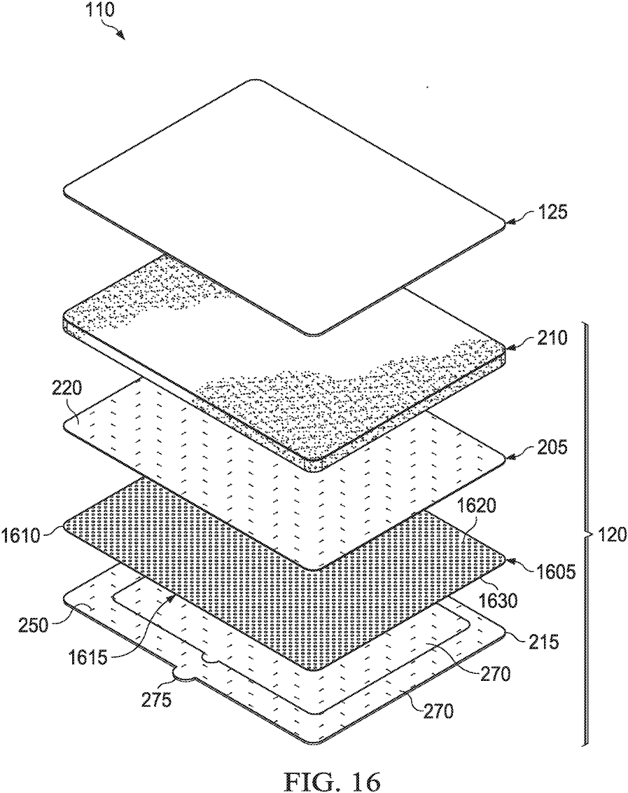

FIG. 16 is an assembly view of another example of the dressing 110 of FIG. 1, illustrating additional details that may be associated with some embodiments in which the tissue interface 120 may comprise additional layers. In the example of FIG. 16, the tissue interface 120 comprises a fifth layer 1605, in addition to the first layer 205 and the second layer 210, and the third layer 215, but does not include the fourth layer 400 of the embodiment of FIG. 4. In some embodiments, the fifth layer 1605 may be adjacent to the first layer 205 opposite the second layer 210. The fifth layer 1605 may also be bonded to the first layer 205 in some embodiments.

The fifth layer 1605 may comprise or consist essentially of a sealing layer formed from a soft, pliable material, such as a tacky gel, suitable for providing a fluid seal with a tissue site, and may have a substantially flat surface. For example, the fifth layer 1605 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. The fifth layer 1605 may include an adhesive surface on an underside and a patterned coating of acrylic on a top side. The patterned coating of acrylic may be applied about a peripheral area to allow higher bonding in regions that are likely to be in contact with skin rather than the wound area. In other embodiments, the fifth layer 1605 may comprise a low-tack adhesive layer instead of silicone. In some embodiments, the fifth layer 1605 may have a thickness between about 200 microns (µm) and about 1000 microns (µm). In some embodiments, the fifth layer 1605 may have a hardness between about 5 Shore OO and about 80 Shore OO. Further, the fifth layer 1605 may be comprised of hydrophobic or hydrophilic materials.

In some embodiments, the fifth layer 1605 may be a hydrophobic-coated material. For example, the fifth layer 1605 may be formed by coating a porous material, such as, for example, woven, nonwoven, molded, or extruded mesh with a hydrophobic material. The hydrophobic material for the coating may be a soft silicone, for example.

The fifth layer 1605 may have corners 1610 and edges 1615. The fifth layer 1605 may include apertures 1620. The apertures 1620 may be formed by cutting or by application of local RF or ultrasonic energy, for example, or by other suitable techniques for forming an opening. The apertures 1620 may have a uniform distribution pattern, or may be randomly distributed on the fifth layer 1605. The apertures 1620 in the fifth layer 1605 may have many shapes, including circles, squares, stars, ovals, polygons, slits, complex curves, rectilinear shapes, triangles, for example, or may have some combination of such shapes.

Each of the apertures 1620 may have uniform or similar geometric properties. For example, in some embodiments, each of the apertures 1620 may be circular apertures, having substantially the same diameter. In some embodiments, the diameter of each of the apertures 1620 may be between about 1 millimeter and about 50 millimeters. In other embodiments, the diameter of each of the apertures 1620 may be between about 1 millimeter and about 20 millimeters.

In other embodiments, geometric properties of the apertures 1620 may vary. For example, the diameter of the apertures 1620 may vary depending on the position of the apertures 1620 in the fifth layer 1605. The apertures 1620 may be spaced substantially equidistant over the fifth layer 1605. Alternatively, the spacing of the apertures 1620 may be irregular.

As illustrated in the example of FIG. 16, some embodiments of the dressing 110 may include the third layer 215, which may protect the fifth layer 1705 prior to use and cover an adhesive 1630 applied to a surface of the fifth layer 1605. As in other embodiments, at least one of the plurality of separable regions 270 of the third layer 215 may be removed so as to expose at least a portion of the adhesive 1630 on the fifth layer 1605.

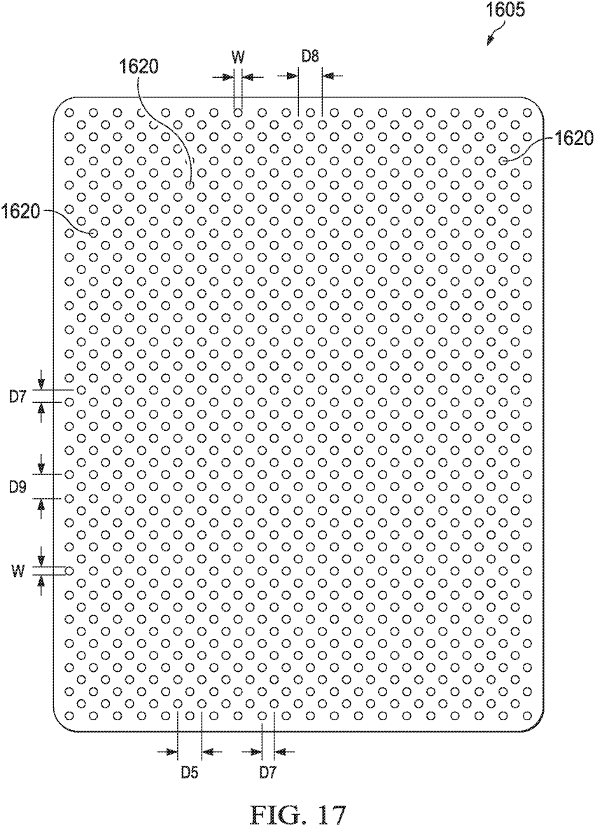

FIG. 17 is a schematic view of an example configuration of the apertures 1620, illustrating additional details that may be associated with some embodiments of the fifth layer 1605. In some embodiments, the apertures 1620 illustrated in FIG. 17 may be associated only with an interior portion of the fifth layer 1605. In the example of FIG. 17, the apertures 1620 are generally circular and have a width W, which may be about 2 millimeters in some examples. FIG. 17 also illustrates an example of a uniform distribution pattern of the apertures 1620. In FIG. 17, the apertures 1620 are distributed across the fifth layer 1605 in a grid of parallel rows and columns. Within each row and column, the apertures 1620 may be equidistant from each other, as illustrated in the example of FIG. 17. The rows may be spaced a distance D7, and the apertures 1620 within each of the rows may be spaced a distance D8. For example, a distance D7 of about 3 millimeters on center and a distance D8 of about 3 millimeters on center may be suitable for some embodiments. The apertures 1620 in adjacent rows may be aligned or offset. For example, adjacent rows may be offset, as illustrated in FIG. 17, so that the apertures are aligned in alternating rows separated by a distance D9. A distance D9 of about 6 millimeters may be suitable for some examples. The spacing of the apertures 1620 may vary in some embodiments to increase the density of the apertures 1620 according to therapeutic requirements.

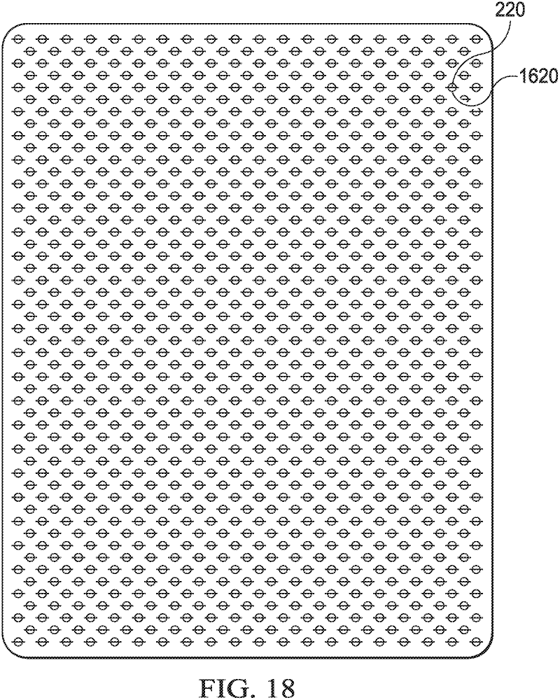

FIG. 18 is a schematic view of the fifth layer 1605 of FIG. 16 overlaid on the first layer 205 of FIG. 3, illustrating additional details that may be associated with some example embodiments of the tissue interface 120. For example, as illustrated in FIG. 18, the perforations 220 may be aligned, overlapping, in registration with, or otherwise fluidly coupled to the apertures 1620 in some embodiments In some embodiments, one or more of the perforations 220 may be registered with the apertures 1620 only in an interior portion, or only partially registered with the apertures 1620. The perforations 220 in the example of FIG. 18 are generally configured so that each of the perforations 220 is registered with only one of the apertures 1620. In other examples, one or more of the perforations 220 may be registered with more than one of the apertures 1620. For example, any one or more of the perforations 220 may extend across two or more of the apertures 1620. Additionally or alternatively, one or more of the perforations 220 may not be registered with any of the apertures 1620.

As illustrated in the example of FIG. 18, the apertures 1620 may be sized to expose a portion of the first layer 205, the perforations 220, or both through the fifth layer 1605. In some embodiments, one or more of the apertures 1620 may be sized to expose more than one of the perforations 220. For example, some or all of the apertures 1620 may be sized to expose two or three of the perforations 220. In some examples, the length of each of the perforations 220 may be substantially equal to the diameter of each of the apertures 1620. More generally, the average dimensions of the perforations are substantially similar to the average dimensions of the apertures 1620. For example, the apertures 1620 may be elliptical in some embodiments, and each of the perforations 220 may have a length L that is substantially equal to the major axis or the minor axis of the ellipse. In some embodiments, the dimensions of the perforations 220 may exceed the dimensions of the apertures 1620, and the size of the apertures 1620 may limit the effective size of the perforations 220 exposed through the fifth layer 1605.

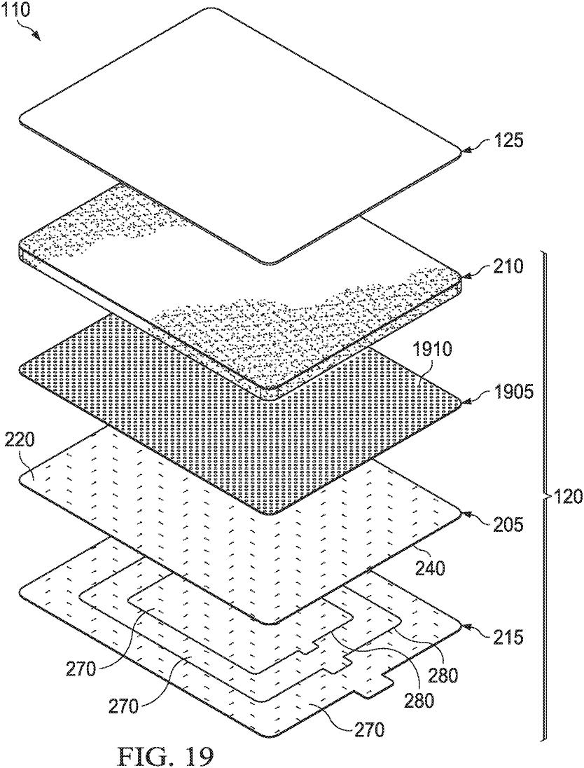

FIG. 19 is an assembly view of another example of the dressing 110, illustrating additional details that may be associated with some example embodiments of the therapy system of FIG. 1. In the example of FIG. 19, the tissue interface 120 comprises a tie layer 1905 in addition to the first layer 205 and the second layer 210. The tie layer 1905 may have perforations 1910 and may have a thickness between 10 microns and 100 microns in some embodiments. The tie layer 1905 may be clear, colored, or printed. As illustrated in FIG. 19, the tie layer 1905 may be disposed between the first layer 205 and the second layer 210. The tie layer 1905 may also be bonded to at least one of the first layer 205 and the second layer 210 in some embodiments.

The tie layer 1905 may comprise polyurethane film, for example, which can be bonded to the first layer 205 and the second layer 210. For example, if the first layer 205 is formed of a polyethylene film and the second layer 210 is polyurethane foam, the first layer 205 may be more readily bonded to the tie layer 2005 than directly to the second layer 210.

In the embodiment of FIG. 19, the first layer 205 may have the adhesive 240 thereon. The third layer 215 may be in contact with the adhesive 240. As in other embodiments, the third layer 215 includes the plurality of separable regions 270 that may be separated by perforations 280. One or more of the plurality of separable regions 270 may be removed to expose at least a portion of the adhesive 240 so that the dressing 110 can be adhered to the periwound during instillation therapy.

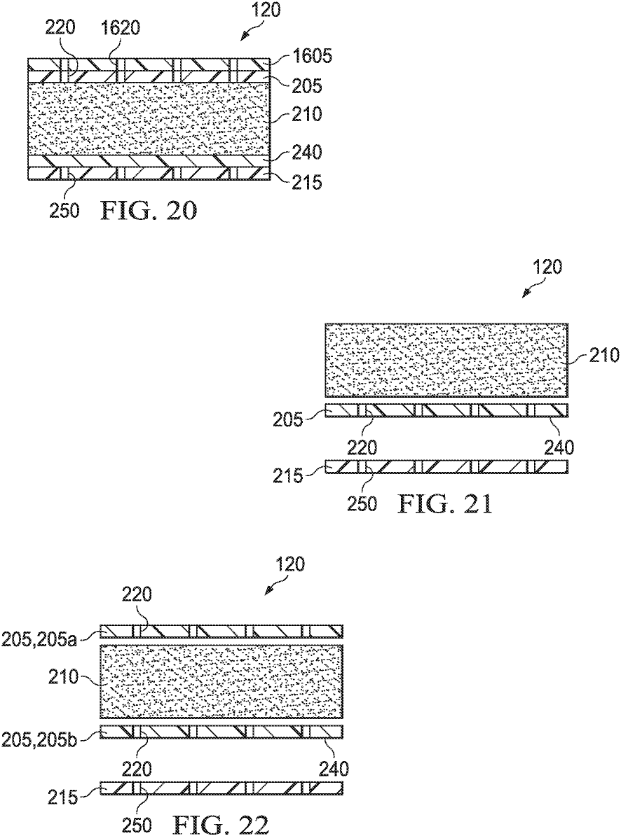

FIG. 20 is a side, cross-sectional view of another example of the tissue interface 120, illustrating additional details that may be associated with some example embodiments of the therapy system of FIG. 1. In some embodiments, as shown in FIG. 20, the dressing 110 may include the second layer 210, which is a manifold layer. The second layer 210 may have an adhesive 240 applied on one side thereof. The first layer 205 may be on a second side of the second layer 210, opposite the adhesive 240. The first layer 205 may be a polymer film including the plurality of perforations 220. A perforated silicone, polyurthethane gel, or acrylic layer, such as the fifth layer 1605 may be on a second side of the first layer 205. The third layer 215 may cover the adhesive 240. The adhesive 240 may be an acrylic adhesive or a silicone adhesive

FIG. 21 is an exploded side, cross-sectional view of another example of the tissue interface 120, illustrating additional details that may be associated with some example embodiments of the therapy system of FIG. 1. As shown in FIG. 21, an embodiment of the tissue interface 120 may include the second layer 210, the first layer 205, and the third layer 215. The first layer 205 may include the adhesive 240 opposite the second layer 210. In some embodiments, the adhesive 240 may be an acrylic adhesive. In some embodiments, the adhesive 240 may be a silicone adhesive. In some embodiments, the adhesive 240 may be a polyurethane gel adhesive.

FIG. 22 an exploded side, cross-sectional view of another example of the tissue interface 120, illustrating additional details that may be associated with some example embodiments of the therapy system of FIG. 1. As shown in FIG. 22, an embodiment of the tissue interface 120 may include the second layer 210 between two first layers 205, shown as 205a and 205b, where a first one 205a of the first layers 205 is on a first side of the second layer 210 and a second one 205b of the first layers 205 is on a second side of the second layer 210 opposite the first side. The first one 205a of the first layers 205 may not include any adhesive opposite the second layer 210. The second one 205b of the first layers 205 may include the adhesive 240 opposite the second layer 210. In some embodiments, the adhesive 240 may be an acrylic adhesive. In some embodiments, the adhesive 240 may be a silicone adhesive. In some embodiments, the adhesive 240 may be a polyurethane gel adhesive. Additionally, the tissue interface 120 may include the third layer 215 proximate to the adhesive 240. With the inclusion of a first layer 205 on either side of the second layer 210, the first one 205a of the first layers 205 not having any outward-facing adhesive and the second one 205b of the first layers 205 having adhesive 240, the tissue interface 120 can be flipped, as desired, so that the adhesive 240 does or does not face the tissue site. Additionally, none, some, or all of the third layer 215 may be removed to selectively expose the adhesive 240 to the tissue site.

In some embodiments, one or more of the components of the dressing 110 may additionally be treated with an antimicrobial agent. For example, the second layer 210 may be a foam, mesh, or non-woven coated with an antimicrobial agent. In some embodiments, the second layer 210 may comprise antimicrobial elements, such as fibers coated with an antimicrobial agent. Additionally or alternatively, some embodiments of the first layer 205 may be a polymer coated or mixed with an antimicrobial agent. In other examples, the fluid conductor 290 may additionally or alternatively be treated with one or more antimicrobial agents. Suitable antimicrobial agents may include, for example, metallic silver, PHMB, iodine or its complexes and mixes such as povidone iodine, copper metal compounds, chlorhexidine, or some combination of these materials.

Additionally or alternatively, one or more of the components may be coated with a mixture that may include citric acid and collagen, which can reduce bio-films and infections. For example, the second layer 210 may be foam coated with such a mixture.

In use, the third layer 215 may be at least partially removed to expose the adhesive 240, the adhesive 285, or both, which can provide a lower surface of the dressing 110 to be placed within, over, on, or otherwise proximate to a tissue site, particularly a surface tissue site and adjacent epidermis. The third layer 215 may be at least partially removed to expose at least a portion of the adhesive 240 so as to adhere the dressing 110 to the periwound and protect the periwound from maceration risk during instillation therapy.

The geometry and dimensions of the tissue interface 120, the cover 125, or both may vary to suit a particular application or anatomy. For example, the geometry or dimensions of the tissue interface 120 and the cover 125 may be adapted to provide an effective and reliable seal against challenging anatomical surfaces, such as an elbow or heel, at and around a tissue site.

Additionally or alternatively, instillation solution or other fluid may be distributed to the dressing 110, which can increase the pressure in the tissue interface 120. The increased pressure in the tissue interface 120 can create a positive pressure differential across the perforations 220 in the second layer 210, which can open the perforations 220 to allow the instillation solution or other fluid to be distributed to the tissue site. The adhesive 240 can seal the perforations 220 if in contact with an attachment surface, such as epidermis, which can prevent exposure of instillation solution to the attachment surface. Otherwise, the adhesive 240 allows movement of instillation solution through the perforations 220.

In some embodiments, when the adhesive is not required or desired, the dressing 110 may be flipped so that a non-adhesive film layer or the liner is left in place on the film layer so that the dressing 110 does not adhere to the wound and periwound. Thus, the user may select to not have any adhesive on the area under the manifold in contact with the wound or to have adhesive expose to adhere to the periwound. In some embodiments, the dressing 110 may provide for different adhesives in different areas of the dressing 110. For example, a portion of the third layer 215 may remain in contact with a portion the first layer 205, such that the portion of the third layer 215 covers the adhesive 240 on the portion of the first layer 205. Additionally, radially about this portion of the first layer 205 an acrylic adhesive may be used where maceration may be a concern. This may be accomplished, for example, by removing a portion of the third layer 215 exposing the adhesive 240. Additionally, a region of silicone adhesive may extend radially about the adhesive 240 to achieve a fluid and/or air seal with the tissue site. Accordingly, in some embodiments, concentric regions of no adhesives and/or variations of adhesives may be utilized.

The systems, apparatuses, and methods described herein may provide significant advantages. For example, some embodiments of the dressing 110 allow for the selective application and regions of adhesive between the perforated film layer 205 and the periwound, which may reduce or prevent maceration of the periwound if the dressing 110 is used with instillation therapy. Additionally, the perforated release liner 215 may be kept in place on the dressing 110 or removed depending on the application, allowing the dressing 110 to be with or without instillation therapy.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context.

Features, elements, and aspects described in the context of some embodiments may also be omitted, combined, or replaced by alternative features serving the same, equivalent, or similar purpose without departing from the scope of the invention defined by the appended claims. For example, one or more of the features of some layers may be combined with features of other layers to provide an equivalent function. For example, in some configurations the dressing 110, the container 115, or both may be separated from other components for manufacture or sale. In other example configurations, components of the dressing 110 may also be manufactured, configured, assembled, or sold independently or as a kit.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A dressing for treating a tissue site with instillation therapy, the dressing comprising:
a first layer comprising a polymer film having a plurality of passages through the polymer film;
a second layer adjacent to the first layer, the second layer having a plurality of apertures;
an adhesive layer on at least a portion of the first layer; and
a third layer on the adhesive layer, the third layer being at least partially removable from the adhesive layer.

2. The dressing of claim 1, wherein the third layer is not adhesive.

3. (canceled)

4. The dressing of claim 1, wherein the passages comprise a plurality of fenestrations.

5. The dressing of claim 1, wherein the third layer includes a plurality of regions, the plurality of regions being separable.

6. The dressing of claim 5, wherein the plurality of regions are configured in a tessellate pattern or in concentric rings.

7. (canceled)

8. (canceled)

9. The dressing of claim 1, wherein the third layer includes at least one pull tab.

10. The dressing of claim 1, wherein the adhesive layer comprises a polyurethane adhesive, an acrylic adhesive or a silicone adhesive.

11. (canceled)

12. The dressing of claim 1, wherein:
the second layer is a manifold comprising a first surface and a second surface opposite the first surface; and
the first layer is adjacent to the first surface.

13. The dressing of claim 12, further comprising:
a fourth layer adjacent the second surface of the second layer, the fourth layer comprising a polymer film having a plurality of fluid restrictions through the polymer film.

14. The dressing of claim 13, further comprising:
a fifth layer adjacent to the fourth layer, the fifth layer comprising a polymer drape.

15. (canceled)

16. The dressing of claim 12, further comprising:
a fourth layer adjacent the second surface of the second layer, the fourth layer comprising a gel having a coat weight of about 250 grams per square centimeter.

17-31. (canceled)

32. The dressing of claim 1, wherein the passages comprise or consist essentially of elastomeric valves in the polymer film, and the elastomeric valves are normally closed.

33. The dressing of claim 1, wherein the passages comprise one or more of fenestrations, slits, and intersecting slits in the polymer film.

34. (canceled)

35. (canceled)

36. (canceled)

37. (canceled)

38. A system for treating a tissue site, comprising:
a dressing, the dressing including:
a first layer comprising a polymer film having a plurality of fluid restrictions through the polymer film;
a second layer adjacent to the first layer, the second layer comprising a polymer having a plurality of apertures;
an adhesive layer on at least a portion of the first layer; and
a third layer on the adhesive layer, the third layer being at least partially removable from the adhesive layer; and
a source of instillation solution.

39. The system of claim 38, wherein the third layer is a non-adhesive third layer.

40. (canceled)

41. The system of claim 38, wherein the third layer includes a plurality of fenestrations.

42. The system of claim 38, wherein the third layer includes a plurality of regions, the plurality of regions being separable.

43-48. (canceled)

49. The system of claim 38, wherein the second layer is a manifold comprising a first surface and a second surface opposite the first surface, the first layer adjacent the first surface.

50. The system of claim 38, further comprising:
a fourth layer adjacent a second side of the second layer, the fourth layer comprising a polymer film having a plurality of fluid restrictions through the polymer film.

51. (canceled)

52. The system of claim 38, further comprising:
a fourth layer adjacent a second side of the second layer, the fourth layer comprising a gel.

53-55. (canceled)
